(19) United States
(12) Patent Application Publication
(10) Pub. No.: US 2010/0184727 A1
(43) Pub. Date: Jul. 22, 2010

(54) TREATMENT OF EXCESS SEBUM PRODUCTION

(75) Inventors: Alan Geoffrey Roach, Oxfordshire (GB); Paul Goldsmith, Oxfordshire (GB)

Correspondence Address: HESLIN ROTHENBERG FARLEY & MESITI PC, 5 COLUMBIA CIRCLE, ALBANY, NY, 12203, US

(73) Assignee: SUMMIT (CAMBRIDGE) LIMITED, Abingdon, Oxfordshire (GB)

(21) Appl. No.: 12/303,680

(22) PCT Filed: Jun. 7, 2007
(86) PCT No.: PCT/GB07/02098

§ 371 Date: Oct. 29, 2009

(30) Foreign Application Priority Data
Jun. 7, 2006 (GB) 0611240.3

(51) Int. Cl. A61K 31/60; A61K 31/46; A61K 31/4725; A61K 31/40; A61K 31/352; A61K 31/216; A61K 31/222; A61K 31/02; A61K 31/327; A61K 31/20; A61K 31/192; A61K 31/465; A61P 17/10; A61P 17/08; A61P 31/00; A61P 31/10

(52) U.S. Cl. 514/161; 514/278; 514/291; 514/304; 514/305; 514/424; 514/428; 514/454; 514/534; 514/546; 514/743; 514/714; 514/558; 514/569; 514/159; 514/162; 514/163; 514/355

(57) ABSTRACT

A muscarinic receptor antagonist is useful for the treatment or prevention of a condition associated with excess sebum production or excretion.

### FIELD OF THE INVENTION

This invention relates to the treatment of excess sebum production and/or excretion from sebaceous glands.

### BACKGROUND OF THE INVENTION

Sebum is an oily secretion from sebaceous glands in the skin and serves many functions. Sebum is involved in the development epidermal structure and maintains an epidermal permeability barrier, as well as transporting anti-oxidants to the surface of the skin and providing protection from microbial colonisation. An increased rate of sebum excretion is termed seborrhoea. Seborrhoea is a feature of many conditions including Parkinson's disease.

Seborrhoeic dermatitis is characterised by the appearance of red, flaking, greasy areas of skin, most commonly on the scalp, nasolabial folds, ears, eyebrows and chest. Seborrhoeic dermatitis of the scalp is often referred to as dandruff and may range from mild scalp flaking to pronounced dense, diffuse, adherent scale on the scalp. In the clinical literature seborrhoeic dermatitis may be also referred to as “sebopsoriasis”, “seborrhoeic eczema”, “dandruff” and “pityriasis capitis”.

There now appears general agreement that yeast infections are a causative factor in seborrhoeic dermatitis. The yeast consumes the specific saturated fatty acids necessary for their proliferation leaving high concentrations of unsaturated fatty acids on the skin. It has been suggested that penetration of the modified sebaceous secretions result in inflammation, irritation and flaking of the skin. Therefore whilst the primary treatment may focus around controlling fungal levels, this does not address the entire problem. It would be highly desirable to control and decrease sebum production.

Treatments for seborrhoeic dermatitis include antifungal agents such as zinc pyrithione, cinnamic acid, azoles, cyclopirox, terbinafine, as well as non-specific topical agents such as selenium sulphide/sulphur, tar, lithium succinate, benzoyl peroxide, propylene glycol, and corticosteroids. Corticosteroids are effective topical anti-inflammatory treatments but have severe systemic side effects. Most compounds are given topically although if topical antifungals prove ineffective then agents such as ketoconazole, itraconazole or terbinafine can be given orally. The efficacy of itraconazole and terbinafine may be attributed to the excretion of these agents in sebum.

It is accepted in the clinical community that acne vulgaris is accompanied with clinical seborrhea and there is a direct relationship between the sebum excretion rate and the severity of acne vulgaris. Although sebum production increases during adolescence (particularly in boys, because of androgen stimulation), increased sebum alone does not cause acne. Bacteria, most importantly P. acnes, are present in increased numbers in persons who have acne. Much of the inflammation that eventually occurs arises from the action of enzymes produced by the bacteria.

Acne is divided into three categories comedonal, inflammatory and nodulocystic acne. Diagnosis of the condition into these categories determines the treatment schedule. Mild to moderate forms of comedonal and inflammatory acne are treated topically. Mild but significant comedonal acne responds well to topical retinoids (adapalene, tretinoin and isotretinoin) or benzoyl peroxide. Comedonal acne with inflammatory lesions is currently treated with topical antibiotics and antimicrobial agents (clindamycin, erythromycin, tetracycline, and azelaic acid). Moderate to severe inflammatory and nodulocystic acne are generally treated with oral antibiotics and retinoids. Severe side effects are associated with high dose retinoids and therefore treatment with these agents is often limited.

There is a need for new therapies that reduce sebum excretion rates which are well tolerated for the treatment of seborrhoea in Parkinson's patients as well as producing effective combination treatments for seborrhoeic dermatitis and acne vulgaris.

Glycopyrrolate and scopolamine are older generation anti-muscarinic drugs and block all five muscarinic receptor subtypes and centrally-mediated side effects have been reported, particularly with scopolamine. The older generation non-selective compounds are a mixture of compounds that enter the brain, and those that contain a quaternary ammonium function and therefore do not readily penetrate the blood brain barrier. Examples of compounds that contain a quaternary amine function are propantheline, methscopolamine, homatropine methylbromide, poldine, ipratropium, trospium and glycopyrrolate.

In recent years, a newer generation of muscarinic receptor antagonists, which possess some selectivity for and preferentially block muscarinic M3 receptors relative to the other M receptor subtypes have been introduced or are currently being developed for the treatment of bladder disorders. Examples of newer M3 muscarinic compounds, but not limited to, are darifenacin, solifenacin, fesoterodine, and zamifenacin. Oxybutynin is not a new generation muscarinic antagonist but it displays modest activity for M1 and M3 receptors over the other subtypes. It is used extensively to treat overactive bladder disorders. However, compounds with differing selectivity profiles at muscarinic receptors have been developed to treat COPD.

### SUMMARY OF THE INVENTION

The present invention is use of a muscarinic receptor antagonist for the manufacture of a medicament for the treatment or prevention of a condition associated with excess sebum production or excretion.

### DESCRIPTION OF THE PREFERRED EMBODIMENTS

Preferred muscarinic receptor antagonists for use in the invention are darifenacin, solifenacin, tolterodine, fesoterodine, zamifenacin, oxybutynin, revatropate, Ro-3202904 (PSD-506), propantheline, methscopolamine, homatropine, methyibromide, poldine, ipratropium, tiotrpium, trospium or glycopyrrolate. Preferential M3 muscarinic receptor antagonists such as darifenacin, solifenacin, tolterodine, fesoterodine, zamifenacin, oxybutynin, revatropate and Ro-3202904 (PSD-506), are particularly preferred. Oxybutynin is further preferred. The advantages of using a preferential M3 muscarinic receptor antagonist is that they do not cause the extensive side-effects associated with blockade if all the muscarinic receptors, such as sedation, dry mouth and hypotension.

Examples of conditions associated with excess sebum production are seborrhoea, seborrhoeic dermatitis or acne vulgaris.

For the treatment of seborrhoeic dermatitis, the or each active agent may be administered together with antifungals or antiproliferatives such as zinc pyrithione, cinnamic acid, azoles (such as clotrimazole, econazole, ketoconazole, miconazole or sulconazole), cyclopirox, terbinafine, selenium sulphide, coal tar, lithium succinate, benzoyl peroxide, propylene glycol or a corticosteroid, given topically to affected areas.

For the treatment of acne vulgaris, the or each active agent maybe administered together with benzoyl peroxide, azelaic acid, adapalene, tretinoin, isotretinoin, salicyclic acid, nicotinamide, a histone deacetylase inhibitor (such as valproic acid) or an antibacterial agent (such as tetracycline, erythromycin or clindamycin), given topically to affected areas. The or each active agent may also be administered with retinoid (such as isotretinoin), an anti-androgen (such as cyproterone or ethinylestradiol), a histone deacetylase inhibitor (such as valproic acid) or an antibiotic agent (such as tetracycline, oxytetracycline, doxycycline, minoxycycline, erythromycin or trimethoprim), given orally.

Each active agent may be used, according to the invention, in any appropriate form, e.g. as a salt, hydrate or prodrug. If a chiral molecule, it may be used as a racemate, as a non-racemic mixture or as a substantially single enantiomer.

In general, each active agent either alone or in combination may be administered by known means, in any suitable formulation, by any suitable route. Each active agent, either alone or in combination, is preferably administered topically or orally. For topical administration, it is preferably formulated as a solution, gel, lotion, cream, shampoo or a spray. For oral administration, it is preferably formulated as a tablet, troche, lozenge, capsule, emulsion, syrup or elixir.

The respective reactive agents may be formulated together in a single dosage form. Alternatively, they may be formulated separately and packaged together, or they may be administered independently.

Compositions for use in the invention may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.1-99% by weight of active compound. The compositions of the invention are generally prepared in unit dosage form. Preferably, a unit dose comprises the active ingredient in an amount of 0.01 to 100 mg. The excipients used in the preparation of these compositions are the excipients known in the art.

Appropriate dosage levels may be determined by any suitable method known to one skilled in the art. It will be understood, however, that the specific dose level for any particular patient will depend upon a variety of factors including the age, body weight, general health, sex, diet, time of administration, route of administration, rate of excretion, drug combination. Preferably, the active compound is administered at a frequency of 1 to 4 times per day.

Compositions for topical administration are suitable for use in the invention. The pharmaceutically active compound or combination of compounds may be dispersed in a pharmaceutically acceptable cream, lotion, shampoo, ointment or gel. A suitable cream may be prepared by incorporating the active compound in a topical vehicle such as light liquid paraffin, dispersed in an aqueous medium using surfactants. An ointment may be prepared by mixing the active compound with a topical vehicle such as a mineral oil or wax. A gel may be prepared by mixing the active compound with a topical vehicle comprising a gelling agent. Topically administrable compositions may also comprise a matrix in which the active compound is dispersed, so that the compound is held in contact with the skin, in order to administer the compound or combinations of compounds to the skin.

Compositions for oral administration include known pharmaceutical forms for such administration, for example tablets, troches, lozenges, aqueous or oily suspensions, dispersible tablets, powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, polyoxyethylene hydrogenated castor oil, fatty acids such as oleic acid, or in a mineral oil such as liquid paraffin or in other surfactants or detergents. The oily suspensions may contain a thickening agent, for example beeswax, hard paraffin or ‘0 cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid, find use in the preparation of injectables.

The following study provides evidence of utility of the present invention.

### Study 1

The study examined the effects of oxybutynin on sebum production in 9 healthy male volunteers. This was double-blind, randomised, placebo-controlled dose study

The subjects were assessed on each dosing occasion for sebum production pre-dose and 1 h, 2.5 h, 4 h and 6 h post-dose. Skin sebum excretion rates were determined using a Sebumeter® (Optical measurement of sebum excretion using opalescent film imprint, 2006, Handbook of non-invasive methods and the skin, Second Edition). Vital signs were recorded at specified times during each study period and adverse events were reported throughout.

For each subject, the maximum % reduction in sebum production compared to placebo was calculated for each dose level. Using this information, a mixed effects regression analysis of the % reduction in sebum versus dose with subject as a random effect was performed.

### Results

The results of the regression analysis are shown in the table below.

Mixed Model Regression Analysis

| | | Maximum % | | | | | |
|---|---|---|---|---|---|---|---|
| | | Reduction | | | P-value^{a} for | ED30 | ED50 |
| Parameter | Dose | LS Means | Intercept | Slope | slope | (mcg) | (mcg) |
| Sebum | 1 mg | 21.59 | 22.95 | −10.07 | 0.0002 | 5.26 | 7.25 |
| Production | 2 mg | −16.94 | | | | | |
| | 5 mg | −34.74 | | | | | |
| | 10 mg | −75.52 | | | | | |

^{a}5% significance level

The results show that sebum production decreased with dose, following administration of 2 mg, 5 mg and 10 mg oxybutynin, an effect that was significant with increasing dose levels.

### Study 2

The anaerobic cutaneous propionibacteria e.g. Propionibacterium acnes are implicated in the pathogenesis of acne vulgaris. The test organism Propionibacterium acnes is classified as a ‘Type’ strain for the species. This study examined the minimum inhibitory concentration (MIC) and, minimum bactericidal concentration (MBC) of P. acnes NCTC 737 to oxybutynin chloride and darifenacin hydrobromide, both muscarinic acetylcholine receptor antagonists.

MIC determinations were carried out using a standard 96-well plate broth micro-dilution assay. Wilkins-Chalgren anaerobe broth at pH 6 and pH 7.1±0.2 was used for P. acnes. The MIC range for the test compounds was 1.95-1000 mg/L in doubling dilutions, the diluting solvent acted as the positive control. The test range for the control compounds varied depending on known activity levels. Following the MIC assay the MBC was determined by sub-culturing 5 μl of the first well that shows positive growth and all the subsequent wells indicating no growth on to Wilkins-Chalgren (pH 6 or pH 7.1±0.2) agar plates.

Benzoyl peroxide and erythromycin were used as control compounds in P. acnes assays. Benzoyl peroxide and erythromycin were both dissolved in ethanol.

### Results

The results of the study are shown in the tables below.

TABLE 1
MIC and MBC date of test compounds and controls at pH 6

| Test Compound | MIC (mg/L) ≠ | MBC (mg/L) ≠ |
|---|---|---|
| Oxybutynin chloride | 500 | 1000 |
| Darifenacin hydrobromide | 500 | 1000 |
| Benzoyl peroxide | 31.25 | 62.5 |
| Erythromycin | 0.63 | >2.5 |

≠ Mean value from a minimum of 4 replicates
ZoI—Zone of Inhibition

Test assays indicate that oxybutynin chloride and darifenacin hydrobromide demonstrate some moderate antimicrobial activity against P. acnes at pH 6.

TABLE 2
MIC and MBC data of test compounds
and controls at pH 7.1 ± 0.2

| Test Compound | MIC (mg/L) ≠ | MBC (mg/L) ≠ |
|---|---|---|
| Oxybutynin chloride | 500 | 500 |
| Darifenacin hydrobromide | 500 | 500 |
| Benzoyl peroxide | 31.25 | 62.5 |
| Erythromycin | 0.313 | >2.5 |

≠ Mean value from a minimum of 4 replicates
ZoI—Zone of Inhibition

Test assays again indicate that oxbutynin chloride and darifenacin hydrobromide demonstrate some moderate antimicrobial activity against P. acnes at pH 7.1±0.2.

1.-12. (canceled)

13. A method for the treatment or prevention of a condition associated with excess sebum production or excretion comprising administering an effective amount of a muscarinic receptor antagonist to a subject.

14. The method of claim 13 wherein the antagonist is selected from: darifenacin, solifenacin, tolterodine, fesoterodine, zamifenacin, oxybutynin, revatropate, Ro-3202904 (PSD-506), propantheline, methscopolamine, homatropine, methylbromide, poldine, ipratropium, tiotropium, trospium and glycopyrrolate.

15. The method of claim 13 wherein the antagonist is administered topically.

16. The method of claim 15 wherein the antagonist is formulated as a solution, gel, lotion, cream, shampoo or a spray.

17. The method of claim 13 wherein the antagonist is administered orally.

18. The method of claim 17 wherein the antagonist is formulated as a tablet, troche, lozenge suspension, dispersible tablet, powder or granules, emulsion, capsules, syrup or elixir.

19. The method of claim 13 wherein the condition is selected from: seborrhoea, seborrhoeic dermatitis and acne vulgaris.

20. The method of claim 19 wherein the condition is selected from seborrhoea or seborrhoeic dermatitis and wherein the antagonist is administered topically and the patient treated is also administered an antifungal or an anti proliferative.

21. The method of claim 19 wherein the condition is acne vulgaris and wherein the antagonist is administered topically and the patient treated is also administered benzoyl peroxide, azelaic acid, adapalene, a retinoid, salicyclic acid, nicotinamide, a histone deacetylase inhibitor or an antibacterial agent.

22. The method of claim 19 wherein the condition is acne vulgaris and wherein the antagonist is administered orally and the patient treated is also administered a retinoid, an anti-androgen, a histone deacetylase inhibitor or an antibiotic agent.
